(19) United States
(12) Patent Application Publication
(10) Pub. No.: US 2014/0107459 A1
(43) Pub. Date: Apr. 17, 2014

(54) DEVICES, SYSTEMS, AND METHODS FOR INTRAOCULAR MEASUREMENTS

(71) Applicant: ALCON RESEARCH, LTD., Fort Worth, TX (US)

(72) Inventors: Casey Jean Lind, Orange, CA (US); Mikhail A. Ovchinnikov, Dana Point, CA (US); Robert Joseph Sanchez, JR., Oceanside, CA (US)

(73) Assignee: Alcon Research, Ltd., Fort Worth, TX (US)

(21) Appl. No.: 13/649,323

(22) Filed: Oct. 11, 2012

(51) Int. Cl. A61B 3/16

(52) U.S. Cl. 600/403; 600/398

(57) ABSTRACT

An implantable sensor assembly sized for insertion within an eye of an average human is disclosed. The sensor assembly comprises a sensor and an antenna system coupled to the sensor, wherein the antenna system is pliable between an expanded condition and an unexpanded condition, and the expanded condition has a predetermined shape configured to interface with tissue within the eye in a manner that stabilizes the sensor assembly.

### BACKGROUND

Glaucoma, a group of eye diseases characterized by an increase in intraocular pressure (TOP), is one of the leading causes of blindness worldwide. Most forms of glaucoma result when the IOP increases to pressures above normal for prolonged periods of time, thereby adversely affecting the retina and optic nerve. IOP can increase due to high resistance to the drainage of the aqueous humor relative to its production. Left untreated, an elevated IOP causes irreversible damage to the optic nerve and retinal fibers resulting in a progressive, permanent loss of vision. Thus, monitoring the intraocular pressure is crucial to proper treatment and evaluation of glaucoma.

FIG. 1 is a diagram of the front portion of an eye 10 that helps to explain the processes of glaucoma. In FIG. 1, representations of the lens capsule 110, the lens 115, cornea 120, iris 130, ciliary body 140, zonules 145, trabecular meshwork 150, and Schlemm's canal 160 are pictured. The lens capsule 110, located behind the iris 130, contains the lens 115, which is seated between an anterior capsule 162 and a posterior capsule 164. The anterior capsule 162 and the posterior capsule 164 meet at an equatorial region 166 of the lens capsule 110. The lens 115 (or an artificial intraocular lens (IOL)) may be acted on by the zonular forces exerted by the zonules 145 extending from the ciliary body 140 and surrounding the periphery of the lens capsule 110. The zonules 145 and the ciliary body 140 anchor the lens capsule 110 in place and facilitate accommodation, the process by which the eye changes optical power to maintain a clear focus on an image as its distance varies.

Anatomically, an anterior chamber 170 and a posterior chamber 175 include the structures that cause elevated IOP which may lead to glaucoma. The eye's ciliary body 140, which lies beneath the iris 130 and adjacent to the lens capsule 110, continuously produces aqueous humor, the clear fluid that fills the anterior segment of the eye (the space between the cornea 120 and lens capsule 110). The aqueous humor flows out of the anterior chamber 170 (the space between the cornea 120 and his 130) through the trabecular meshwork 150 and the uveoscleral pathways (not shown), both of which contribute to the aqueous drainage system located in the angle of the anterior chamber 170. The trabecular meshwork 150, which is commonly implicated in glaucoma, extends circumferentially around the anterior chamber 170 and appears to act as a filter limiting the outflow of aqueous humor and providing a back pressure that directly relates to IOP. Schlemm's canal 160 is located beyond the trabecular meshwork 150, and is fluidically coupled to collector channels (not shown) allowing aqueous humor to flow out of the anterior chamber 170. The two arrows in FIG. 1 show the flow of aqueous humor from the ciliary body 140, over the lens capsule 110, over the iris 130, through the trabecular meshwork 150, into Schlemm's canal 160 and its collector channels. The delicate balance between the production and drainage of aqueous humor determines the eye's IOP.

In general, glaucoma therapy today consists of monitoring and regulating the IOP by either medical or surgical interventions. An important aspect of treating glaucoma includes frequent monitoring of the IOP. The IOP of normal people varies throughout the day. Abnormal pressure elevations may occur at odd hours, e.g., very early in the morning, or when it is impractical for the patient or healthcare provider to measure and record IOPs. Such diurnal curves, which measure individual IOP measurements over time, are considered to be of great value in the diagnosis and treatment of glaucoma, and to evaluate the response to glaucoma therapy. Thus, frequent monitoring is necessary to obtain an accurate assessment of a patient's average intraocular pressure.

Measurement of IOP is commonly done with an external tonometer in a physician's office. The need to have a healthcare professional available during these IOP measurements and the risks of corneal abrasion, reactions to topical anesthetics, and the transmission of infectious agents limit the accessibility and ease of monitoring intraocular pressure in glaucoma patients. Other disadvantages of these external IOP measurements include their cost, the disruption to the patient's normal activities, and possible interference with the measured IOP caused by artificial changes to natural sleep patterns and/or repeated manipulation of the corneal surface (i.e., the tonography effect). Moreover, IOP readings using external devices are a function of both the IOP value and the corneal stiffness, which reduces the accuracy of the IOP reading.

The devices, systems, and methods disclosed herein overcome one or more of the deficiencies of the prior art.

### SUMMARY

In one exemplary embodiment, the present disclosure describes an implantable sensor assembly sized for insertion within an eye of a patient, comprising a sensor and an antenna system coupled to the sensor. In one aspect, the antenna system is pliable between an expanded condition and an unexpanded condition, and the expanded condition has a predetermined shape configured to interface with tissue within the eye in a manner that stabilizes the sensor assembly.

In some embodiments, the antenna system comprises an antenna surrounded by a casing.

In some embodiments, the sensor assembly further comprises a haptic coupled to the antenna system, wherein the haptic is shaped and configured to stabilize the sensor assembly within the eye.

In another exemplary embodiment, the present disclosure describes a sensor assembly system for measuring characteristics within an eye of a patient, the sensor assembly comprising a sensor, an antenna system coupled to the sensor, and a delivery instrument configured to position the sensor assembly in the eye. In one aspect, the antenna system is configured to self-expand into a predetermined shape configuration and is sized to stabilize the sensor assembly in the eye. In one aspect, the delivery instrument comprises a lumen and a plunger longitudinally disposed within the lumen. The lumen is sized to receive the sensor assembly and the plunger is configured to translate longitudinally within the lumen to engage the sensor and displace the sensor assembly from the lumen.

In another exemplary embodiment, the present disclosure describes a method for positioning a sensor assembly relative to an eye. The method comprises inserting the sensor assembly in an unexpanded condition into a lumen of a delivery instrument sized to receive the sensor assembly, wherein the sensor assembly comprises a sensor coupled to an antenna system configured to self-expand, and wherein the delivery instrument comprises a plunger longitudinally disposed within a lumen and configured to selectively engage the sensor. The method further comprises moving the plunger along the longitudinal axis of the lumen toward a distal end of the delivery instrument to displace the sensor assembly from the lumen of the delivery instrument into the eye.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is a side view diagram of the front portion of an eye.

FIG. 2 illustrates a top plan view of an exemplary sensor assembly according to one embodiment of the present disclosure.

FIG. 3 illustrates a side view of the sensor assembly shown in FIG. 2.

FIG. 4 illustrates a side view of an exemplary sensor assembly according to another embodiment of the present disclosure.

FIGS. 5 and 6 illustrates side views of the sensor assembly shown in FIG. 2 being ejected from an exemplary delivery instrument and inserted into an eye according to one exemplary embodiment of the present disclosure.

FIG. 7 illustrates a partially cutaway perspective view of the sensor assembly shown in FIG. 2 positioned within an eye according to one exemplary embodiment of the present disclosure.

FIG. 8 illustrates a cutaway side view of the sensor assembly shown in FIG. 2 positioned in an unexpanded condition within an exemplary delivery instrument including an exemplary plunger tip according to one embodiment of the present disclosure.

FIG. 9 illustrates a cutaway side view of the sensor assembly shown in FIG. 2 emerging from the exemplary delivery instrument shown in FIG. 8.

FIG. 10 illustrates a cutaway side view of the sensor assembly shown in FIG. 2 in an expanded condition upon emerging from the exemplary delivery instrument shown in FIG. 8.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately. For simplicity, in some instances the same reference numbers are used throughout the drawings to refer to the same or like parts.

The present disclosure relates generally to sensor assemblies and associated delivery systems and methods. In some embodiments, the sensor assemblies are configured for use in ophthalmic conditions requiring frequent or constant monitoring of intraocular pressure (IOP), such as, by way of non-limiting example, glaucoma. In some embodiments, the sensor assemblies are configured for use in ophthalmic conditions requiring frequent or constant monitoring or measurement of other ocular characteristics, including, by way of non-limiting example, measurement of the anterior chamber pressure, measurement of capsular tension or strain (e.g., the pressures or forces exerted by ciliary zonules), and/or measurement of the Ph levels or other chemical characteristics of the anterior chamber. In some instances, embodiments of the present disclosure may be configured to be part of an ophthalmic surgical system.

In one exemplary aspect, the present disclosure provides a sensor assembly utilizing a sensor coupled to an antenna having shape memory characteristics. The sensor assembly may assume an unexpanded condition to facilitate atraumatic insertion into and removal from an eye through a primary incision, and can assume a predetermined, expanded condition within the eye. In one embodiment, in its expanded condition, the sensor assembly comprises a substantially circular ring antenna with an attached microsensor. In some embodiments, the sensor assembly includes substantially pliable engaging portions referred to here as haptics that support the sensor assembly against internal structures of the eye, allowing the sensor assembly to be self-stabilized and self-retained in the eye (i.e., without the use of sutures, tacks, or a manually held instrument). Therefore, the sensor assembly disclosed herein allows for constant monitoring of a patient's IOP or other ocular characteristics, thereby facilitating the diagnosis, the treatment, and/or the monitoring of the progression of various eye conditions. The present disclosure also provides an inserter that may be used to insert the sensor assembly. In some embodiments, the sensor assembly may be inserted into the eye during a phacoemulsification procedure through the same incision that was used to insert an IOL.

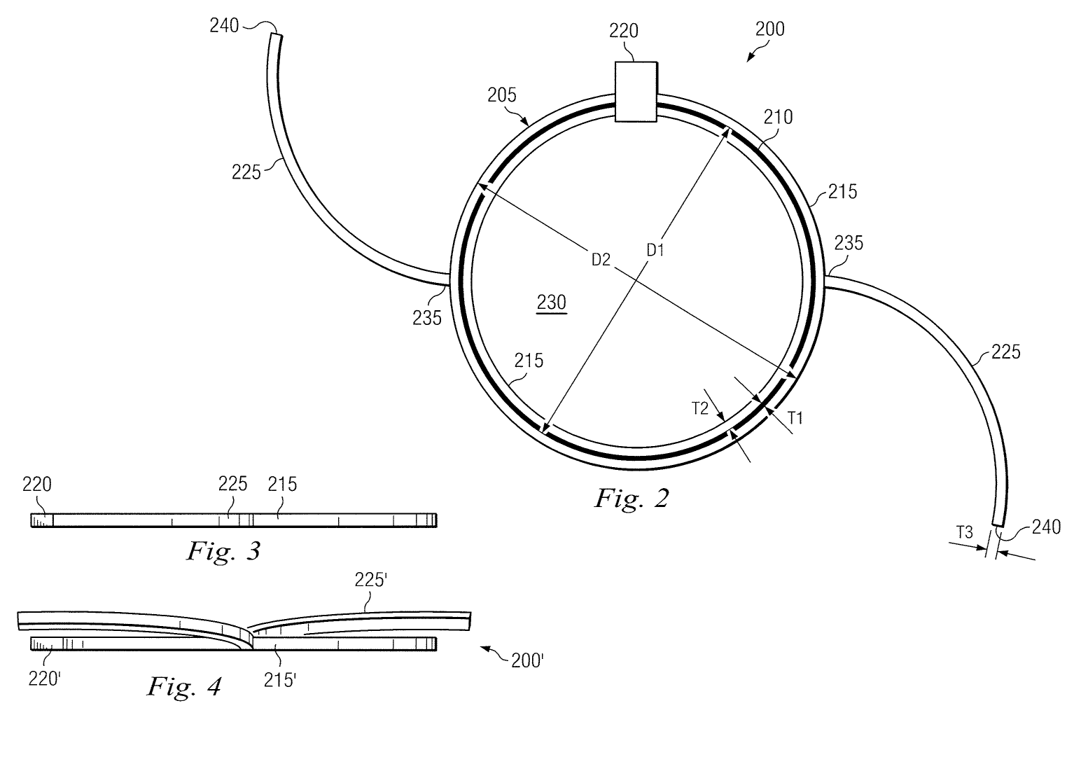

FIG. 2 illustrates a sensor assembly 200 in an expanded condition according to one embodiment of the present disclosure. Though the sensor assembly 200 shown in FIG. 2 is configured for use in the eye, the sensor assembly may be used in other anatomical systems, including by way of non-limiting example, the gastrointestinal system, the respiratory system, and the cardiovascular system.

The sensor assembly 200 comprises an antenna system 205 having an antenna 210 surrounded by a casing 215 and comprises a sensor 220. In the pictured embodiment, the sensor assembly 200 further comprises support members or haptics 225 extending from the casing 215.

In the pictured embodiment, the antenna system 210 comprises a conductive circular ring or toroid. The antenna 210 can be considered a loop or annular antenna having a central opening 230. In some embodiments, the antenna 210 comprises a radiofrequency (RF) antenna.

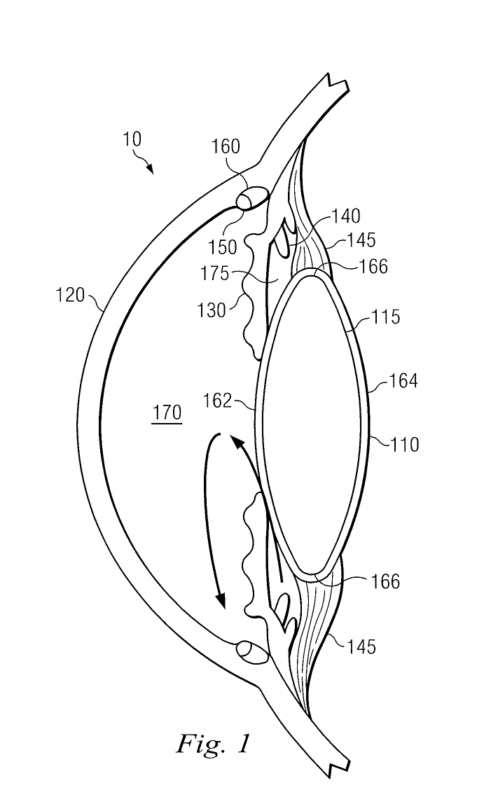

The antenna 210 is expandable from an unexpanded condition to an expanded condition having a predetermined shape configuration. For example, in the embodiment pictured in FIG. 2, the antenna 210, in an expanded condition, comprises a continuous, closed, annular ring with a predetermined circular shape that substantially corresponds to the contours of the equatorial region (e.g., 166 in FIG. 1) of an average human lens capsule. In other embodiments, the antenna may have any of a variety of predetermined shapes in the expanded condition, including, by way of non-limiting example, an annular, closed, generally circular shape, an open, generally circular shape, and an annular, polygonal shape. In some embodiments, the antenna comprises an open shape such as, by way of non-limiting example, an open ring or a C-shaped ring. For example, in some embodiments, the predetermined shape may be an oval, an elliptical, or a polygonal, or a horseshoe shape. In the pictured embodiment, the antenna 210 has a substantially circular cross-sectional shape. In other embodiments, the antenna may have any of a variety of cross-sectional shapes, including without limitation, rectangular, ovoid, square, rhomboid, and crescent.

The antenna 210 is constructed from a structurally deformable biocompatible material that can elastically or plastically deform without compromising its integrity. The antenna 210 may be made from or coated with a self-expanding biocompatible material. Examples of the antenna material include conductive materials such as, by way of non-limiting example, Nitinol, an elastically compressed spring temper biocompatible material, copper, silver, platinum, gold, and alloys of similar conductive materials. Examples of the antenna coating materials or composite coatings include, by way of non-limiting example, silicone, polyimide, peek, polypropylene, parylene or similar materials. Other materials having shape memory characteristics, such as particular metal alloys, may also be used. The shape memory materials allow the antenna to be restrained in a low profile configuration during delivery into the eye and to resume and maintain its expanded shape in vivo after the delivery process. The material composition of the antenna 210 resiliently biases the antenna toward the expanded condition. In particular, in this example, the antenna is formed of an elastic material allowing the antenna to elastically deform to an unexpanded state to facilitate delivery through a small incision (e.g., through a tubular delivery instrument), and spring back to an expanded state as it enters the eye. In other embodiments, the antenna may be made of a shape memory alloy having a memory shape in the expanded configuration. In other embodiments the antenna may be comprised of a conductive material, with a shape memory alloy or a material with a spring back characteristics coupled to it in a non-conductive manner.

The antenna 210 may be sized to have an external diameter D1 ranging from, for example only, approximately 3 to 6 mm in an expanded condition to provide adequate stabilization in the eye and communication capabilities while remaining small enough to limit interference with other surgical instruments and/or implants, such as, by way of non-limiting example, an IOL. The antenna may be sized to have a cross-sectional diameter or thickness T1 ranging from, for example only, approximately 0.25 mm to 2 mm. In some embodiments, the antenna includes a thickness T1 greater than its height, which may range from, for example only, approximately 50 to 100. Other diameter and thickness ranges are contemplated. In some embodiments, the diameter D1 matches the diameter of a capsular bag in an average human eye.

The casing 215 is a tubular sleeve completely encasing the antenna 210. The casing 215 may be formed of any of a variety of structurally deformable biocompatible materials that can elastically or plastically deform without compromising integrity, including, by way of non-limiting example, flexible polymers such as polytetrafluoroethylene (PTFE), silicone, silicone polyimide, polycarbonate, polymethylmethacrylate (PMMA), nylon, prolene, polyurethane, silastic, polyamide or a combination thereof, or any other biocompatible material having the requisite properties of resilience and flexibility. The casing 215 may be sized to have a cross-sectional diameter or thickness T2 ranging from, for example only, approximately 0.9 to 100 μm, although other sizes are contemplated. In combination, the antenna 210 and casing 215 is sized to have an external diameter D2 comprising D1+(2×T2)+T1. In combination, the antenna 210 and casing 215 may be sized to have an external diameter D2 ranging from, for example only, approximately 3.25 to 8.0 mm in an expanded condition to provide adequate stabilization in the eye while remaining small enough to limit interference with other surgical instruments and/or implants, such as, by way of non-limiting example, an IOL. Other diameter ranges are contemplated. In some embodiments, the diameter D2 matches the diameter of a capsular bag in an average human eye.

The sensor 220 is fixedly disposed on or coupled to the antenna system 205, and the sensor 220 extends radially from the antenna 205. In other embodiments, the sensor may be removably coupled to the antenna 205. In some embodiments, the sensor is coupled to the antenna 210. In some embodiments, the sensor 220 may consist of a microchip sensor, such as, for example, a pressure sensor microchip that changes the antenna response as a function of pressure. In some embodiments, the sensor includes microelectromechanical systems (MEMS) technology. In some embodiments, the sensor assembly 200 may include an electronic circuit such as an integrated circuit chip with a sensor component for interaction with optic, electromagnetic, sonic, or other energy forms. In some embodiments, the sensor 220 is configured to measure pressure, such as, for example, the pressure within the anterior chamber 170. Such sensors may assist in the determination of IOP when coupled to an accessory and/or external device. In some embodiments, the sensor 220 is configured to measure pH levels or other chemical characteristics of the anterior chamber 170. In some embodiments, the sensor 220 is configured to measure the temperature within the anterior chamber 170. Such temperature measurements may allow the user and/or the sensor assembly to calibrate for temperature variations in the sensor measurements. In some embodiments, the sensor 220 is configured to measure capsular tension, or forces exerted by the zonules 145 on the lens capsule 110 (shown in FIG. 1).

In the pictured embodiment, the sensor assembly 200 includes only one sensor 220 fixedly coupled to the antenna 220. In other embodiments, the sensor assembly may include any number and arrangement of sensors that allow for adequate measurement and/or monitoring of ocular characteristics. The number and arrangement of the sensors 220 may be selected in consideration of, among other factors, the type of characteristic(s) to be measured, the patient's medical condition, and/or locations at which sensors are typically placed for particular measurements, inclusive of redundancy to prevent or avoid single point failure.

In some embodiments, the sensor 220 is integrally formed with the antenna. In other embodiments, the sensor assembly 200 comprises a multi-component device with the sensor 220 attached to the antenna 210 and/or the casing 215 by any of a variety of attachments mechanisms, including one or more of an adhesive, a threaded engagement, a snap-fit engagement, a frictional engagement, over-molding, heat-shrinking, heat welding, and/or any other mechanism for fixedly coupling the sensor 220 and the antenna 210. In some embodiments, the sensor 220 is removeably coupled to the antenna.

In the pictured embodiment, the sensor assembly 200 includes two haptics 225, which are shaped and configured to allow the sensor assembly to be self-supporting and self-stabilizing within the eye when in an expanded condition. In other embodiments, the sensor assembly may include any number and arrangement of haptics that allow for adequate self-stabilization of the sensor assembly 200 within the eye. In other embodiments, the antenna 210 and the casing 215, in combination and without haptics, are shaped and configured such that the sensor assembly is self-supporting and self-stabilizing within the eye when in an expanded condition. The haptics 225 comprise substantially pliable, curved, elongate members extending outwardly from the casing 215. In FIG. 2, the haptics 225 are shown in an expanded condition where the haptics flare outward from the casing 215. The haptics 225 include a root end 235 adjacent the casing 215 and a free end 240. In the pictured embodiment, the arc traversed by the haptics 225 is substantially circular and curved toward the casing 215.

In some embodiments, the haptics 225 include spring-like shape memory characteristics such that the haptics can assume an unexpanded condition with the application of force by being offset from the neutral or expanded position (e.g., by being compressed against the casing 215) and subsequently returned to an expanded condition when the force is removed. In particular, the haptics 225 are configured to be easily compressed or constrained and held in such a compressed or unexpanded condition to facilitate insertion into the eye. The resilient haptics 225 are configured to spontaneously return to an expanded, at rest condition, as illustrated in FIG. 2, when released from the compression or constraint to facilitate self-stabilization within the eye. These may return to their at rest condition under elastic forces or by a temperature change when a shape memory material is used. Other embodiments use other forces to return from an unexpanded or compressed condition to an expanded condition. When in an expanded condition, the hoop force of the haptics 225 may assist in stabilizing and/or centering the sensor assembly 200 within the capsular bag of the eye. In some embodiments, the haptics may be spring-loaded haptics.

The haptics 225 can be formed from any of a variety of flexible, nonbiodegradable, and biocompatible materials, such as, by way of non-limiting example, polymethylmethacrylate (PMMA), silicone, silicone polyimide, polycarbonate, nylon, prolene, polyurethane, silastic, polyamide or a combination thereof, or any other biocompatible material having the requisite properties of resilience and flexibility. In other embodiments the haptics may be made from or have a shape memory alloy core. In some embodiments, the haptics 225 are integral extensions of the casing 215, and may be formed from the same shape memory material as the casing 215. In other embodiments, the root ends 235 of the haptics 225 are fixedly attached to the casing 215.

In the pictured embodiment, the haptics 225 extend radially away from the casing 215 to form the farthest periphery of the sensor assembly 200. The haptics are sized and configured such that the haptics can, in an expanded condition, provide adequate stabilization of the sensor assembly within the interior of the eye while remaining small enough to limit interference with other surgical instruments and/or implants. In the pictured embodiment, the haptics 225 have a curved shape configured as non-tapering arcs. In other embodiments, the haptics may have any of a variety of shapes and configurations, including by way of non-limiting example, crescents or teardrops. In some embodiments, the haptic may be tapered along its length from the root end to the free end. Such tapering may provide for a more even distribution of stress across the haptic when the sensor assembly is deployed within the patient. In the pictured embodiment, the haptics 225 have substantially circular cross-sectional shapes. In other embodiments, each haptic may have any of a variety of cross-sectional shapes, including without limitation, rectangular, ovoid, square, rhomboid, and crescent. Each haptic 225 may be sized to have a length (i.e., from the root end 235 to the free end 240) that permits the sensor assembly in an expanded condition to fit within a space having a diameter of up to 12.4 mm. Other diameters are contemplated. Each haptic 225 may be sized to have an external diameter or thickness T3 ranging from, for example only, approximately 0.2 to 3.0 mm. Other length and diameter ranges are contemplated. Although the haptics 225 of the sensor assembly 200 are substantially identical in size and cross-sectional shape, other embodiments may include haptics of varying sizes and shapes.

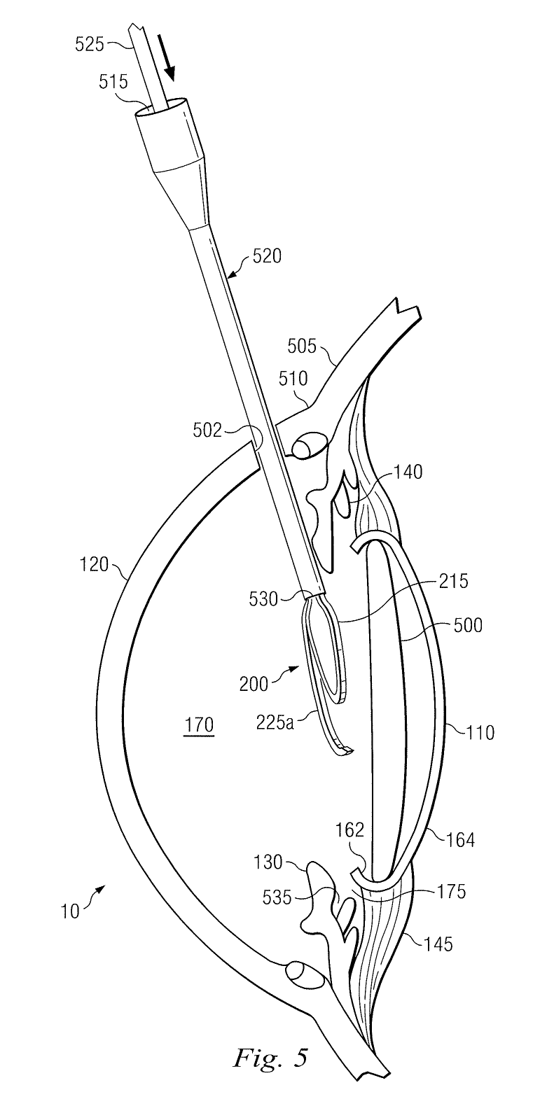

As shown in FIG. 3, the sensor assembly 200 has a substantially planar profile, with both the antenna 210 and the haptics 225 being co-planar. In the embodiment shown in FIG. 4, the sensor assembly 200′ has a slightly non-planer profile due to the haptics 225′ curving away from the antenna 210′ in at least two dimensions and being non-coplanar with the antenna.

In some embodiments, the sensor assembly 200 is shaped and configured to be transparent enough to provide for visualization through the antenna 210, the casing 215, the haptics 225, and/or the sensor 220 to observe, by way of non-limiting example, underlying tissue, vessels, air bubbles, and/or bleeding. In alternate embodiments, the antenna 210, the casing 215, the haptics 225, and/or the sensor 220 are semi-transparent or opaque so as to be clearly visible during ophthalmic procedures. In the pictured embodiment, the external surfaces of the casing 215, the haptics 225, and the sensor 220 are substantially smooth. In other embodiments, the external surfaces of the casing 215, the haptics 225, and/or the sensor 220 may be textured. The various components of the sensor assembly 200 may be coated with any of a variety of biocompatible materials, including, by way of non-limiting example, polytetrafluoroethylene (PTFE).

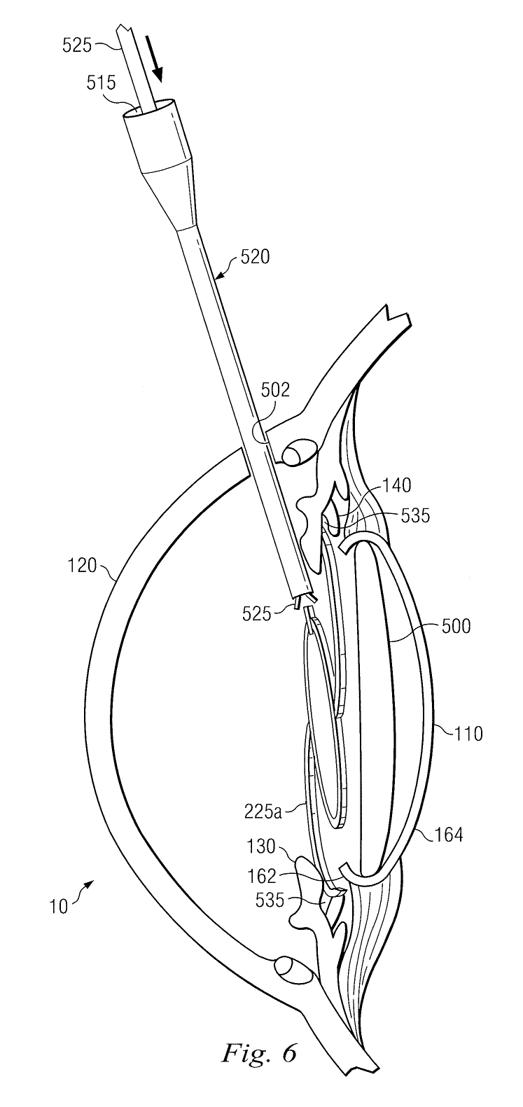

FIGS. 5-6 show a method of inserting the sensor assembly 200 into the eye 10 according to one embodiment of the present disclosure. For the sake of simplicity, the lens capsule 110 is shown post-anterior capsulotomy (i.e., after a portion of the anterior capsule 162 has been removed to facilitate removal of the natural lens and/or insertion of an IOL 500). In some instances, the sensor assembly is inserted into an eye retaining a natural lens, with an artificial IOL. In some instances, the sensor assembly is inserted into an eye retaining a natural lens, without an artificial IOL.

With reference to FIGS. 5 and 6, after a 1.8-4 mm incision 502 is made in the cornea 120, the anterior chamber 170 is filled in a conventional manner with a viscoelastic fluid to prevent the cornea 120 from collapsing and to provide lubrication and support for the subsequent insertion of surgical instruments. In some instances, the incision 502 comprises a clear corneal incision. In other instances, the incision 502 may be made in a sclera 505 or a limbus 510. In some instances, the incision 502 may be the same incision through which an IOL had been inserted into the eye 10. A delivery instrument 520 formed at least in part of a tubular housing is inserted through the incision 502 into the anterior chamber 170. The delivery instrument 520 carries the sensor assembly 200 in an unexpanded condition and has an outer diameter sized to easily pass through the incision 502.

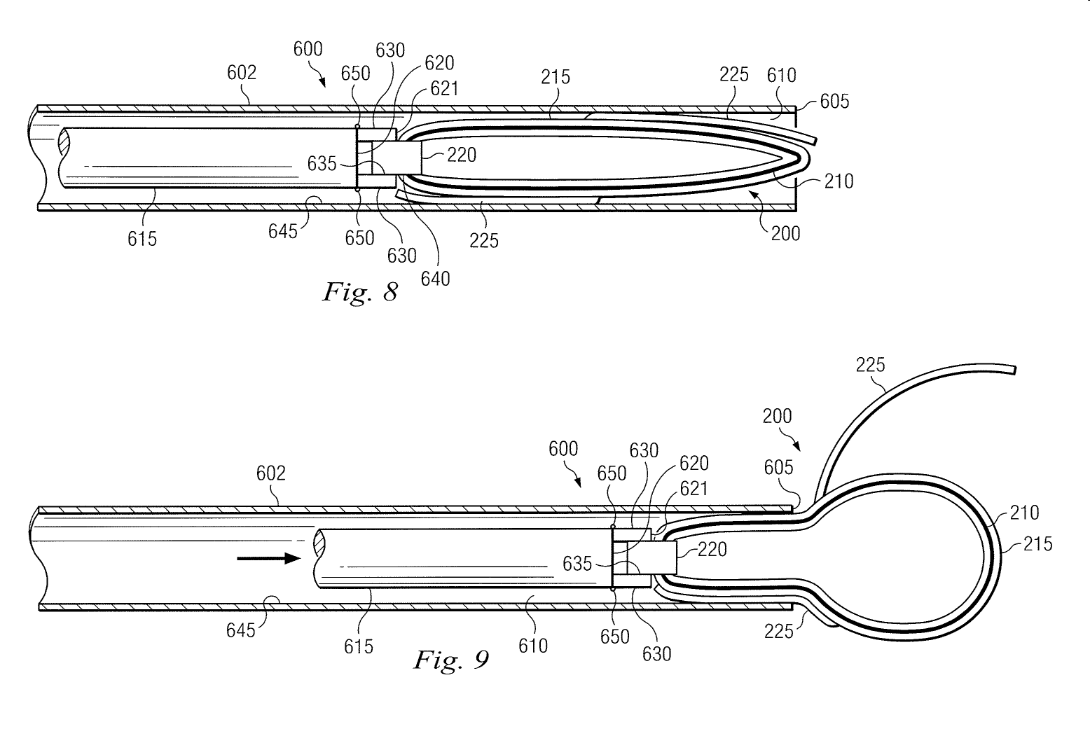

As shown in FIG. 5, as the sensor assembly 200 is passed through a lumen 515 of the delivery instrument 520, the sensor assembly is in an unexpanded condition. In particular, the antenna 210, the casing 215, and the haptics 225 are in an unexpanded condition. In some embodiments, the sensor assembly 200 is delivered into the eye after the insertion of the IOL 500 through the incision 502, and the delivery instrument 520 is the same delivery instrument used to deliver the IOL 500 into the eye 10 immediately prior to the insertion of the sensor assembly 200. In other embodiments, the delivery instrument 520 is a different delivery instrument than the one used to deliver the IOL. In one exemplary method, the user may advance the sensor assembly 200 from the delivery instrument 520 by advancing an actuator or plunger 525 in the direction of the arrow in FIG. 5 through the lumen 515 to push the sensor assembly 200 into the eye 10. The delivery instrument 520 and the plunger 525 are discussed in more detail below with reference to FIGS. 8-10.

As shown in FIGS. 5 and 6, as the sensor assembly 200 emerges from a distal end 530 of the delivery instrument 520, the sensor assembly transitions from the unexpanded configuration into an expanded configuration having a substantially circular shape. In some embodiments, the user may direct the sensor assembly 200 within the eye 10 toward a ciliary sulcus 535, which comprises the space between a posterior surface of the iris 130 and an anterior surface of the ciliary body 140. In one exemplary method, the user may advance the sensor assembly 200 into the posterior chamber 175 to engage a distal-most haptic 225 against the ciliary sulcus 535. In another exemplary method (not shown in FIG. 6), the user may advance the sensor assembly 200 into the anterior chamber 170 to engage and/or embed the distal-most haptic 225a against an anterior surface of the iris 130.

As shown in FIG. 6, after the haptic 225a engages the ciliary sulcus 535, the user may advance the remainder of the sensor assembly 200 from the delivery instrument 520 into the eye 10. The plunger 525 may be utilized to manipulate the sensor assembly 200 to securely position the sensor assembly within the eye 10.

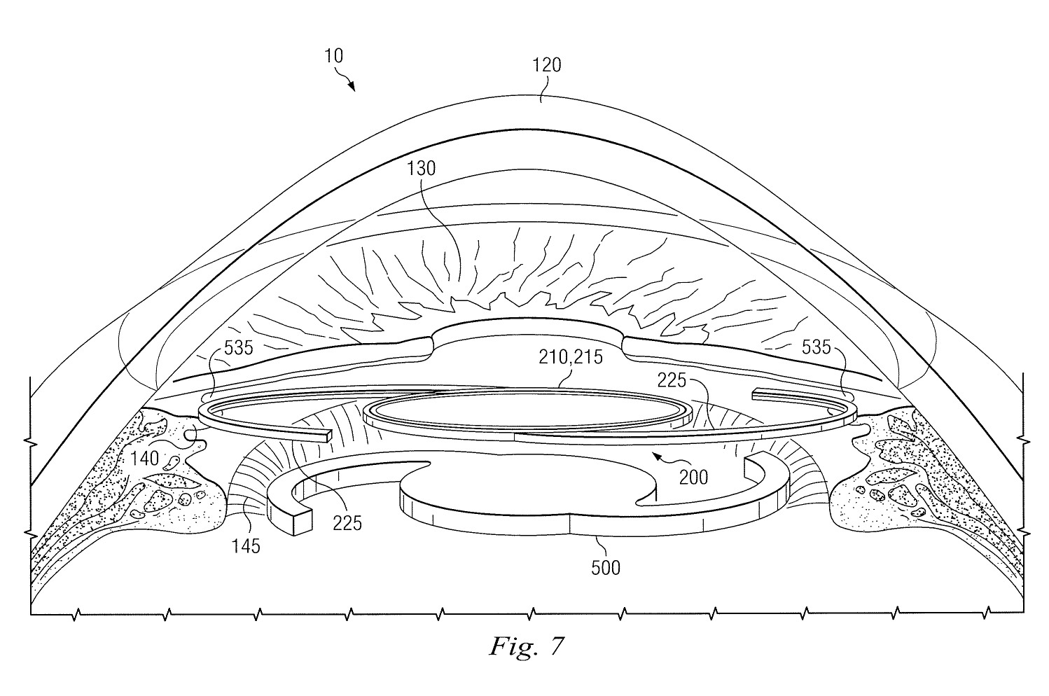

FIG. 7 illustrates the sensor assembly 200 positioned within the eye 10 to perform desired measurement and communication of various pressures and/or chemical characteristics of the eye. After the IOL 500 has been implanted into the eye 10, a surgeon can then implant the sensor assembly 200 through the same incision 502 used to insert the IOL 500 and maneuver the sensor assembly 200 to overlay the IOL 500. As shown in FIG. 7, the haptics 225 are positioned against the ciliary sulcus 535, thereby stabilizing the sensor assembly 200 within the ciliary sulcus 535 and anterior to the implanted TOL 500 without the need for sutures or staples. In some instances, the surgeon may position the sensor assembly 200 such that the antenna 210 resides along the perimeter of the capsular bag just outside the optical edge of the IOL 500. The haptics 225 may aid in centering the sensor assembly 200 within the capsular bag. The sensor assembly 200 is positioned such that it does not obstruct the visual axis. The sensor assembly 200 can wirelessly measure anterior chamber (and/or posterior chamber) characteristics, such as, by way of non-limiting example, chemical features within the chamber, pressures within the chamber, and/or forces exerted by the zonules 145. In other embodiments, as mentioned above, the sensor assembly 200 may be stabilized against an anterior surface of the iris 130, with or without the free ends 240 of the haptics 225 embedded in the iris 130.

FIG. 8 illustrates a schematic view of an exemplary delivery instrument 600 for inserting (and/or removing) the sensor assembly 200 according to one embodiment of the present disclosure. In some embodiments, the delivery instrument 600 is substantially similar to or the same as the delivery instrument 520. As shown in FIG. 8, the delivery instrument 600 comprises a tubular housing 602 including a distal end 605 in communication with a lumen 610. The delivery instrument 600 includes the actuator or plunger 615 longitudinally disposed within the lumen 610. In the pictured embodiment, the sensor assembly 200 is positioned distal to and adjacent the plunger 615 within the lumen 610 in an unexpanded condition.

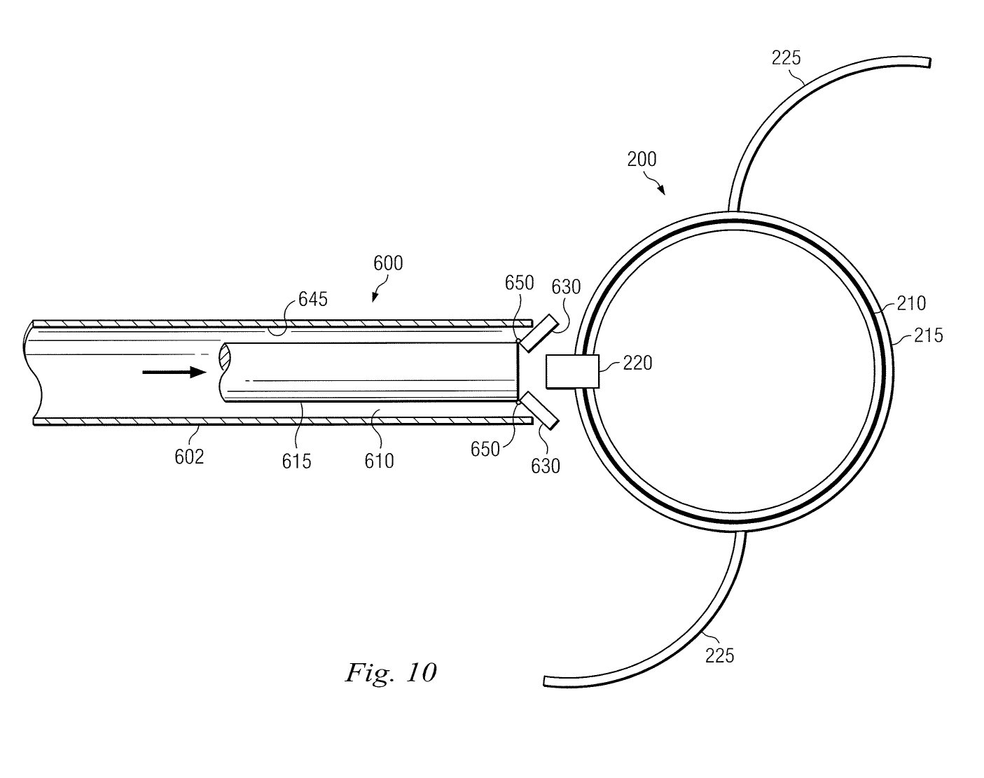

As shown in FIGS. 9 and 10, the delivery instrument 600 is configured so that when the plunger 615 is translated towards the distal end 605 of the delivery instrument 600, the distal plunger end 621 engages and displaces the sensor assembly 200 from the lumen 610, through the distal end 605, and into an eye. As shown in FIG. 9, as the sensor assembly 200 emerges from the distal end 605, the sensor assembly 200 transitions from an unexpanded condition to a more expanded condition in the manner described previously with reference to FIGS. 5 and 6. In some embodiments, the user can manually move the plunger 615 along a longitudinal axis of the housing to displace the sensor assembly 200 from the delivery instrument 600. In other embodiments, the delivery instrument 600 includes an actuating mechanism (not shown) configured to cause longitudinal translation of the plunger along a longitudinal axis of the housing to displace the sensor assembly 200 from the delivery instrument 600.

The plunger 615 is shaped and configured to releasably hold the sensor assembly 200 as the plunger 615 pushes the sensor assembly through the delivery instrument 600. The plunger 615 includes a receiving port 620 that is shaped and configured to engage the sensor 220 as the plunger pushes the sensor assembly 200 through the lumen 610. In the pictured embodiment, the plunger 615 comprises two support arms 630 that are shaped and configured to selectively grasp the sensor 220 within the receiving port 620 while the support arms are within the lumen 610 of the delivery instrument 600. The support arms 630 and the receiving port 620 are shaped and configured to prevent damage to the sensor 220. In some embodiments, the receiving port 620 is configured to surround the sensor 220 and protect the sensor from damage as it is advanced through the delivery instrument 600. In some embodiments, the receiving port 620 is shaped to complement the shape of the sensor 220. In that regard, the support arms 630 can comprise any of a variety of shapes configured to grasp the sensor 220, including, by way of non-limiting example, straight rods, curved arcs, and curvilinear rods, each with or without linear recesses shaped to accommodate portions of the sensor 220. In some embodiments, the support arms 630 are shaped as jaws that can surround more than two surfaces of the sensor 220.

In some embodiments, interior surfaces 635 of the support arms 630 include a sensor engaging feature shaped and configured to releasably couple the plunger 615 to the sensor. In such embodiments, the sensor engaging feature and at least a portion of the sensor 220 are shaped and configured as a mating pair of selectively detachable fasteners. In some embodiments, the sensor engaging feature provides the user with a gripping surface on the support arms 630 that mates with an exterior surface 640 of the sensor 220. The sensor engaging feature and/or the exterior surface 640 may include any of a variety of selectively detachable fasteners such as, by way of non-limiting example, protrusions, indentations, grooves, hooks, and/or loops.

As indicated by FIGS. 8-10, the support arms 630 are shaped and configured to have an unexpanded condition and an expanded condition. For example, in FIGS. 8 and 9, the support arms 630 are shown in an unexpanded condition while within the lumen 610, and in FIG. 10, the support arms 630 are shown in an expanded condition outside the lumen 610. As shown in FIG. 10, as the plunger 615 is advanced toward the distal end 605 of the delivery instrument 600, the support arms 630 transition from an unexpanded condition into an expanded condition upon emerging from the lumen 610. In the pictured embodiment, the support arms 630 are attached to the plunger 615 at joints 650. The joints 650 may include any of a variety of structures, including, by way of non-limiting example, hinges, focal areas of increased flexibility such as compliant mechanisms, and springs. In the embodiment shown in FIGS. 8-10, the support arms 630 comprise spring-loaded elongate members that are biased radially outward from the joints 650 toward an interior wall 645 of the lumen 610. In other embodiments, the support arms may be shaped as detent members configured to securely hold the sensor 220 as it travels through the lumen 610 and then release the sensor 220 when the support arms emerge from the lumen 610. Such support arms may be shaped and configured to securely grasp the sensor 220 within the receiving port 620 by the application of force and/or the use of a sensor engaging feature.

The plunger 615, and in particular the support arms 630, allows the user to manipulate (i.e., position, reposition, remove, and/or otherwise move) the sensor assembly 200 during an ophthalmic procedure while shielding the sensor 220 from damage. The support arms 630, by providing separate contact surfaces around the sensor 220, may function to protect the sensor 220 from damage while the sensor assembly 200 is contained and moved within the lumen 610 of the delivery instrument 600.

Referring to FIG. 9, as the sensor assembly 200 is passed through the lumen 610 of the delivery instrument 600, the sensor assembly 200, and in particular the antenna 210, the casing 215, and the haptics 225, are in an unexpanded condition. In one exemplary method, as described above, the user may advance the sensor assembly 200 from the delivery instrument 600 to engage a distal-most (from the user) haptic 225 against the ciliary sulcus 535 or the iris 130 (shown in FIG. 7).

As indicated by FIGS. 7 and 10, after the haptic 225 engages the ciliary sulcus 535 or the iris 130, the user may advance the remainder of the sensor assembly 200 from the delivery instrument 600 into the eye 10. As shown in FIG. 10, as the sensor assembly 200 emerges from the lumen 610 into the eye 10, the antenna 210 transitions from the unexpanded configuration into an expanded configuration having a substantially circular shape, and the haptics 225 also assume an expanded configuration, flaring outwardly from the casing 215. Moreover, as the sensor assembly 200 emerges from the lumen 610 into the eye 10, the support arms 630 transition from an unexpanded condition to an expanded condition and release the sensor 220. The retention abilities of the support arms 630 may be utilized to manipulate the sensor assembly 200 into a desired position within the eye 10. For example, the user may not advance the support arms 630 of the plunger 615 outside the lumen 610, thereby causing the support arms 630 to assume an expanded condition and release the sensor 220, until the sensor assembly 200 is at least partially positioned in a desired location within the eye. In some instances, the user may utilize the support arms 630 to engage the sensor 220 after its initial release and reposition the sensor assembly 200.

In some instances, when the need for the implanted sensor assembly 200 has concluded, the reverse of the insertion procedure depicted in FIGS. 5 and 6 is carried out. For example, in some instances, the user may use the plunger 615 to grasp the sensor assembly 200 to retract the sensor assembly 200 into a cannula inserted within an eye. The cannula may be substantially similar to the delivery instrument 600. In some instances, the user may use the support arms 630 to grasp the sensor 220 before retracting the sensor assembly 100 into the lumen 610 of the delivery/extraction instrument 600. As the sensor assembly 100 is retracted into the cannula, the sensor assembly 200 transitions from an expanded condition to an unexpanded condition. After retracting the entire sensor assembly 200 into the cannula, the cannula, carrying the sensor assembly 200 in an unexpanded condition, may be withdrawn from the eye.

The various sensor assembly embodiments described herein can utilize a sensor and a flexible antenna to detect and communicate various physiological characteristics, such as, by way of non-limiting example, ocular characteristics. For example, in some embodiments, the sensor assemblies are configured to wirelessly measure anterior chamber (and/or posterior chamber) characteristics, such as, by way of non-limiting example, chemical features within the chamber, temperature, pressures within the chamber, and/or forces exerted by the zonules 145. The implantable sensor assemblies described herein allow a user to wirelessly take different types of measurements (e.g., IOP, zonule forces, capsular tension, temperature, and pH) on a regular basis, without having to visit a healthcare professional or repeatedly manipulate the eye.

It will be clear to one of skill in the art that the sensor assembly embodiments described herein may be altered in various ways without departing from the scope of the invention, and may be used in a variety of non-ocular applications. For example, the sensor 220 may be coated with a medical-grade biocompatible coating prior to being implanted. In some instances, the sensor assembly may be shaped and configured for use in measuring intravascular pressure, pulmonary pressure, biliary-duct pressure, blood pressure, joint pressure, and/or pressure in other fluid-containing bodily tissue. The sensor assemblies may find application in blood pressure monitoring systems, vital signs monitoring systems, chemostasis monitoring systems, respiratory health monitoring systems, and drug delivery systems. In some instances, the sensor assemblies may be incorporated within surgical monitoring equipment, for example where intratubal pressure or chemical readings are required.

The sensor assemblies described herein can assume an unexpanded condition to facilitate atraumatic insertion into and removal from an eye through the same incision used for insertion of a standard IOL, and can assume a predetermined, expanded condition within the eye. Moreover, the various sensor assembly embodiments described herein can stabilize and self-retain their position on an eye and move with the eye as necessary. Although the various sensor assembly embodiments described herein may be used without the aid of a specialized delivery instrument, in some embodiments, the sensor assembly embodiments may be used in conjunction with a specialized delivery instrument utilizing support arms configured to protect the sensor and/or electronics of the sensor assembly during insertion and to provide increased maneuverability of the sensor assembly within the eye. Thus, the delivery instruments and sensor assemblies described herein allow for implanting sensor assemblies and/or MEMS-based assemblies into the eye post-IOL implantation, without damaging the delicate sensor components or requiring an additional incision.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An implantable sensor assembly sized for insertion within an eye of a patient, comprising:
a sensor; and
an antenna system coupled to the sensor, wherein the antenna system is pliable between an expanded condition and an unexpanded condition, the expanded condition having a predetermined shape configured to interface with tissue within the eye in a manner that stabilizes the sensor assembly.

2. The sensor assembly of claim 1, wherein the predetermined shape configuration is selected from a group consisting of an annular, closed, generally circular shape, an open, generally circular shape, and an annular, polygonal shape.

3. The sensor assembly of claim 1, wherein the antenna system comprises a self-expanding biocompatible material.

4. The sensor assembly of claim 3, wherein the antenna system comprises a material having shape memory.

5. The sensor assembly of claim 1, wherein the antenna system comprises an antenna surrounded by a casing.

6. The sensor assembly of claim 5, wherein the casing is composed of a flexible polymer.

7. The sensor assembly of claim 5, wherein the casing includes an external diameter sized to match an internal diameter of a capsular bag in the eye.

8. The sensor assembly of claim 1, wherein the antenna system has an external diameter sized to match an internal diameter of a capsular bag in the eye.

9. The sensor assembly of claim 1, further comprising a haptic coupled to the antenna system, wherein the haptic is shaped and configured to stabilize the sensor assembly within the eye.

10. The sensor assembly of claim 1, further comprising a plurality of haptics coupled to the antenna system, wherein each of the plurality of haptics is shaped and configured to stabilize the sensor assembly within the eye.

11. The sensor assembly of claim 10, wherein the plurality of haptics are disposed symmetrically around the antenna system.

12. The sensor assembly of claim 10, wherein the plurality of haptics include haptics of varying sizes and shapes.

13. The sensor assembly of claim 9, wherein the haptic is configured to conform against an inner margin of ciliary sulcus of the eye.

14. The sensor assembly of claim 9, wherein the haptic is configured to embed within an iris of the eye.

15. The sensor assembly of claim 1, wherein the sensor is fixedly attached to the antenna system.

16. The sensor assembly of claim 1, wherein the sensor is removably coupled to the antenna system.

17. The sensor assembly of claim 1, wherein the sensor includes microelectromechanical systems technology.

18. The sensor assembly of claim 1, wherein the sensor is configured to measure pressure within an anterior chamber of the eye.

19. The sensor assembly of claim 18, wherein the sensor comprises a pressure sensor microchip.

20. The sensor assembly of claim 1, wherein the sensor is configured to measure chemical characteristics within the eye.

21. The sensor assembly of claim 1, wherein the sensor is configured to measure capsular tension within the eye or forces exerted by the zonules of the eye.

22. The sensor assembly of claim 1, wherein the sensor is configured to measure temperature within the eye.

23. A sensor assembly system for measuring characteristics within an eye of a patient, the sensor assembly system comprising:
a sensor assembly comprising:
a sensor; and
an antenna system coupled to the sensor, wherein the antenna system is configured to self-expand into a predetermined shape configuration and is sized to stabilize the sensor assembly in the eye; and
a delivery instrument configured to position the sensor assembly in the eye, the delivery instrument comprising:
a lumen with a longitudinal axis, the lumen sized to receive the sensor assembly; and
a plunger longitudinally disposed within the lumen, the plunger configured to translate longitudinally within the lumen to engage the sensor and displace the sensor assembly from the lumen.

24. The sensor assembly system of claim 23, wherein the predetermined shape configuration is selected from a group consisting of an annular, closed, generally circular shape, an open, generally circular shape, and an annular, polygonal shape.

25. The sensor assembly system of claim 23, wherein the antenna system comprises a material having shape memory.

26. The sensor assembly system of claim 23, wherein the antenna system comprises an antenna surrounded by a flexible casing.

27. The sensor assembly system of claim 26, further comprising a haptic coupled to the casing, wherein the haptic is shaped and configured to stabilize the sensor assembly within the eye.

28. The sensor assembly system of claim 27, wherein the haptic is configured to conform against an inner margin of ciliary sulcus of the eye.

29. The sensor assembly system of claim 27, wherein the haptic is configured to embed within an iris of the eye.

30. The sensor assembly system of claim 23, wherein the sensor is configured to measure pressure within an anterior chamber of the eye.

31. The sensor assembly system of claim 23, wherein the sensor comprises a pressure sensor microchip.

32. The sensor assembly system of claim 23, wherein the plunger includes a plurality of support arms shaped and configured to grasp the sensor and selectively couple the plunger to the sensor assembly.

33. The sensor assembly system of claim 32, wherein the plunger includes a receiving port between the plurality of support arms, the receiving port shaped and configured to seat the sensor.

34. The sensor assembly system of claim 32, wherein at least one of the plurality of support arms include a sensor engaging feature shaped and configured to releasably couple the plunger to the sensor.

35. The sensor assembly system of claim 32, wherein the plurality of support arms include an unexpanded condition and an expanded condition, wherein the plurality of support arms are configured to selectively grasp the sensor in the unexpanded condition and selectively release the sensor in the expanded condition.

36. The sensor assembly system of claim 35, wherein the plurality of support arms are shaped and configured to transition from an unexpanded condition to an expanded condition upon emerging from the lumen of the delivery instrument.

37. The sensor assembly system of claim 23, wherein the antenna system is shaped and configured to self-expand upon emerging from the delivery instrument.

38. A method for positioning a sensor assembly relative to an eye, the method comprising:
inserting the sensor assembly in an unexpanded condition into a lumen of a delivery instrument sized to receive the sensor assembly, wherein the sensor assembly comprises a sensor coupled to an antenna system configured to self-expand, and wherein the delivery instrument comprises a plunger longitudinally disposed within a lumen and configured to selectively engage the sensor; and
moving the plunger along the longitudinal axis of the lumen toward a distal end of the delivery instrument to displace the sensor assembly from the lumen of the delivery instrument into the eye.

39. The method of claim 38, wherein moving the plunger along the longitudinal axis of the lumen toward a distal end of the delivery instrument comprises manually pushing the plunger along the longitudinal axis of the lumen toward a distal end of the delivery instrument.

40. The method of claim 38, wherein moving the plunger along the longitudinal axis of the lumen toward a distal end of the delivery instrument comprises using a mechanical actuator to mechanically translate the plunger along the longitudinal axis of the lumen toward a distal end of the delivery instrument.

41. The method of claim 38, wherein the plunger comprises support arms capable of self-expansion, and moving the plunger along the longitudinal axis of the lumen toward a distal end of the delivery instrument comprises advancing the plunger to selectively engage the sensor with the support arms in an unexpanded condition.

42. The method of claim 41, wherein moving the plunger along the longitudinal axis of the lumen toward a distal end of the delivery instrument to displace the sensor assembly from the lumen of the delivery instrument into the eye further comprises advancing the plunger from the lumen of the delivery instrument to expand the support arms and selectively release the sensor.
